(19) United States
(12) Patent Application Publication
(10) Pub. No.: US 2006/0198830 A1
(43) Pub. Date: Sep. 7, 2006

(54) Ubiquinone composition and a container for its convenient transport and storage

(76) Inventors: Siddharth Shastri, Los Angeles, CA (US); Jarrow L. Rogovin, Los Angeles, CA (US); Kevin M. Connolly, Culver City, CA (US)

Correspondence Address: MCCARTER & ENGLISH LLP;CITYPLACE I, 185 ASYLUM STREET, HARTFORD, CT, 06103, US

(21) Appl. No.: 11/365,539

(22) Filed: Mar. 1, 2006

Related U.S. Application Data
(60) Provisional application No. 60/657,676, filed on Mar. 1, 2005.

(51) Int. Cl. A61K 38/43; A61K 31/12; A61K 36/534; A61K 36/752; A61K 36/23

(52) U.S. Cl. 424/94.1; 424/725; 424/736; 424/747; 514/690

(57) ABSTRACT

The present invention relates to a combination of a hermetically sealed, substantially air-tight container, and a quinone-containing composition disposed therein. The container of the present invention is adapted for the convenient transport, and storage of an effective amount of an active ingredient, for example, ubiquinone, ubiquinol or a mixture thereof, and at least one pharmaceutically acceptable excipient. In another aspect the invention relates to a hermitically sealed container comprising an composition containing a single daily dose of ubiquinone, ubiquinol or both.

### REFERENCE TO RELATED APPLICATION

Under 35 U.S.C. § 119(e) this application claims the benefit of U.S. Provisional Application No. 60/657,676 filed Mar. 1, 2005, which is hereby incorporated by reference in its entirety.

### FIELD OF THE INVENTION

The present invention relates to a nutritional composition comprising an entire daily dose of a quinone, for example, ubiquinone, but substantially avoids the problem of quinone recrystallization. In another aspect the invention relates to a ubiquinone-containing composition with improved solubility, bioavailability, and storage capabilities. In other aspects, the invention relates to a composition comprising an entire daily dose of at least one active ingredient, for example, a ubiquinone and, optionally, at least one pharmaceutically acceptable excipient.

### BACKGROUND OF INVENTION

Ubiquinone or coenzyme Q10 (CoQ10) is an essential antioxidant found in every cell in the body, and is especially concentrated in heart tissue. CoQ10 functions in the mitochondria where it is a critical component of the electron transfer chain, which produces the energy required for a cell's survival. The electron transfer function of CoQ10 is mediated by its ability to undergo cycles of oxidation and reduction within the mitochondrial inner membrane. Due to the action of dehydrogenases within the mitochondria, CoQ10 is efficiently reduced by the acceptance of electrons. It is the ability to subsequently donate electrons or reduce downstream components of the transport chain that results in the critical proton gradient, which mediates the production of cellular energy. This step in the electron transport chain is so critical that the equilibrium is shifted nearly completely to the right in favor of reduced ubiquinone (i.e., ubiquinol). This ensures that the reduction of ubiquinone is not the rate limiting step in energy synthesis. Therefore it is not surprising that essentially all the CoQ10 found in the body is in the reduced form. CoQ10 can only be reduced through the action of dehydrogenase enzymes but once it is reduced CoQ10 also functions as a key antioxidant. Recently it has been shown that reduced CoQ10 is important for the antioxidant activity of vitamin E (tocopherol) in the prevention of lipid peroxidation.

It is estimated that the total CoQ10 content in the body is about 1.5 to about 2 grams. Generally, plasma CoQ10 baseline values range from about 0.60 to 0.80 μg/ml plasma. Assuming a typical individual circulates about 3000 ml of plasma; that means there is approximately 2 mg of CoQ10 in the blood. Only about 3-5 mg is thought to be consumed by humans on a daily basis, mostly from meat and poultry sources. Early studies on CoQ10 indicate that dietary supply contributes 50%, while the other 50% is internally produced. The body normally produces all-trans CoQ10, which can also be produced commercially by microbiological fermentation (Kaneka Q™ brand).

Certain factors accelerate normal metabolic loss of CoQ10. For example, the administration of some pharmaceuticals, such as statins for the lowering of blood cholesterol, can result in up to a 70% CoQ10 reduction in blood plasma, and cause potentially serious side effects. However, because the majority of CoQ10 is not in the blood it is difficult to estimate the amount of CoQ10 that would replenish the losses caused by statins. Some studies suggest that 50-100 mg of CoQ10 per day would be required to counteract a (roughly) 50% drop in plasma CoQ10, and presumably, whole body CoQ10.

Several problems confound the simple administration of CoQ10. The first is that it is relatively insoluble (i.e. hydrophobic), making administration of high doses impractical. Because of CoQ10's low solubility in aqueous solutions and its relatively high molecular weight (863.36 daltons), orally administered CoQ10 is very poorly absorbed. The poor absorption results in a low percentage of the administered dose being bioavailable, even at relatively large doses. For example, studies indicate that administration of 300-1200 mg/day of CoQ10 only results in a plasma CoQ10 concentrations of 3-4 μg/ml.

Next, the oxidized form of CoQ10 is not effective as an antioxidant until it has been reduced. In vivo this requires the interaction of dehydrogenases present only in the mitochondria of cells. Futhermore, the spontaneous oxidation of reduced CoQ10 makes production and storage problematic.

The use of nutritional or dietary supplements to reduce cholesterol is continuing to grow as an alternative to prescription medications. However, currently available CoQ10 supplements are of limited usefulness due to minimal CoQ10 bioavailability related to low absorption, CoQ10 recrystallization, oxidation or a combination of both. In addition, currently available CoQ10 supplements require multiple doses per day, making consumption of adequate dosages problematic.

These shortcomings may be addressed by delivery of a single, solubilized, high-concentration CoQ10 composition. Therefore, there exists an overwhelming need for a CoQ10 supplement that is comprised of natural ingredients that are effective, without the significant side effects which addresses the current shortcomings in the art.

### SUMMARY OF THE INVENTION

The current invention relates to a quinone composition, for example, ubiquinone that is present in a form that provides for adequate bioavailability, eliminates the problem of recrystallization, reduces or eliminates the need to take multiple daily doses as well as prevents the oxidative damage to the nutrient components.

The present invention relates generally to a nutritional composition comprising a quinone compound, for example, ubiquinone or CoQ10, and at least one of an emulsifier, water, additional nutrients, a reducing agent, a solubilizing agent, an oil, a volatile or essential oil, flavoring, excipients, another biologically active agent or combinations thereof.

Emulsifiers utilized in any of the embodiments of the invention include, for example, a phospholipid, for example, a lecithin or phosphotidylcholine; a monoglyceride; casein; gums or modified gums (for example, Arabic, Xanthin, Acacia, or the like); egg yolks; or Phosall 75SA (phophatidylcholine and glyceryl monostearate), with casein and ticamulsion (modified gum arabic); a polyoxyl, for example, Cremophor; medium chain glycerides or combinations thereof. It will be understood by one of ordinary skill in the art that the relative viscosity of the emulsion may be varied by altering the proportions of the various ingredients. As such, emulsions demonstrating a range of potential viscosities are contemplated and should be considered as being within the scope of the present invention.

Solubilizing agents utilized in any of the embodiments of the invention include, for example, an oil, a glyceride or triacylglyceride, volatile or essential oils (for example, terpenes (hemi-, mono-, diterpenesl; for example, the monocyclic monoterpene, limonene, and the like) isolated from peppermint, orange, menthol, spearmint, anise, lemon or mixtures thereof), fibersol-2, maltodextrin, polyoxyethanyl-sitosterol sebacate, polyoxyethanyl-cholesteryl sebacate or polyoxyethanyl-.alpha.-tocopheryl sebacate.

The use of essential oils to increase solubility of an insoluble agent, for example, CoQ10, has been demonstrated in U.S. patent application Ser. No. 10/293,932 (U.S. Patent Pub. No.: US2003/0147927A1) to Khan et al., which is hereby incorporated in its entirety for all purposes. In particular, Khan shows binary mixtures of CoQ10 with essential oils (e.g., peppermint, spearmint, anise, lemon, and menthol) that results in increased solubility of the CoQ10. However, Khan does not show whether bioavailability is increased nor does it show a high-concentration dose of CoQ10 suitable for once-daily administration. While essential oils, for example, lemon or orange, may contain 90-99% limonene some evidence suggests that crude extracts may also contain agents known to cause phototoxicity (e.g., oxidized limonene or limonene-1,2-oxide; furocourmarin; oxypeucedanin; and bergapten). Therefore, in any of the embodiments described herein, the inventors contemplate the use of purified limonene, for example, D-limonene, the major limonene enantiomer produced in nature. The hermetically sealed CoQ10 composition of the invention also provides the addition benefit of essentially eliminating any oxidation of limonene making it particularly useful for topical use.

The reducing agent may be, for example, antioxidants, polyphenols, flavonoids, thiols tocopherols, carotenoids, or water-soluble reductants, for example, riboflavin, glutathione, or ascorbate. In a preferred embodiment, the proportion of reducing agent present is sufficient to result in the reduction of substantially all of the ubiquinone (1, see below) to ubiquinol (2, see below). In an additional aspect, the invention relates to an ubiquinol-containing composition containing a solubilizer present in a suitable volume and concentration to substantially eliminate recrystallization of the ubiquinol, and improve absorption and bioavailability. Non-ionic detergents or surfactants may also be useful in the composition of the invention to increase the bioavailability of the CoQ10. In addition, the nutritional composition of the invention may contain other biologically active agents, nutrients, and/or excipients in addition to CoQ10 at varying ratios. Additional ingredients may be added to provide desired qualities in the product, such as, for example, excipients or additives which will cause the nutritional composition to have an attractive or pleasing taste, consistency, prolong shelf-life or additives that provide additional nutrients, for example, a fatty acid, a lipid, a carbohydrate, an herbal extract, a mineral, a vitamin or any combination thereof.

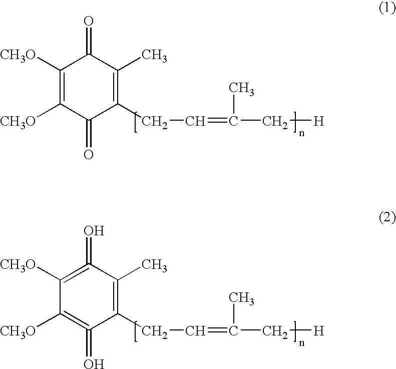

To minimize oxidation and recrystallization of the ubiquinol, the nutritional composition is packaged, stored, and transported in a hermetically sealed container that contains the nutritional composition. The nutritional compositions may be prepared in a substantially air or oxygen free condition, placed in the container, and hermetically sealed to prevent exposure of the nutritional composition to oxygen. The container may be made from a material, such as a foil, which is substantially impermeant to air, and light so that there is no substantial oxidative or UV-induced damage to the CoQ10, thereby substantially extending the mixture's shelf-life and substantially preserving the beneficial properties of the CoQ10. In one embodiment, the volume inside the container is substantially the same as the volume of the nutritional composition contained therein, such that substantially no air spaces exist within the container.

The hermetically sealed container may be sized to contain substantially an entire daily oral dose of CoQ10, obviating the need for an individual to consume excessive quantities of a CoQ10-containing liquid or swallow numerous large capsules several times per day. In one embodiment, the container is substantially sturdy and flexible, for example a plastic bag, or a foil packet such as is used with food condiments. In this form the nutritional composition is substantially more convenient to transport, store, and use. As one of ordinary skill in the art will recognize the hermetically sealed container may be of any suitable material, size, shape, or color, and the above embodiments are given by way of nonlimiting example.

Among the advantages of the invention is that the CoQ10 can be made, stored and transported in a manner which substantially increases the shelf-life of the nutritional composition, and may substantially decrease or eliminate the requirement for the addition of preservatives. Another advantage is that the nutritional composition may provide for easy consumption of an entire daily oral dose. These advantages are given by way of example and are in no way intended to be limiting. Other advantages of the present invention will become apparent to those skilled in the art in view of the following detailed description of preferred embodiments.

### BRIEF DESCRIPTION OF THE DRAWINGS

FIG. 1 shows one embodiments of a hermetically sealed packet containing a ubiquinone composition.

### DETAILED DESCRIPTION OF THE INVENTION

As is used herein, the terms “quinone” and “ubiquinone” are used generally to refer to both their oxidized and reduced forms, except where specifically distinguished. For example, the term “ubiquinol” can be substituted anywhere that “ubiquinone” is used and vice versa, except where the specification makes a clear distinction to one form or the other. Furthermore, the term “quinone” is used in a general sense to include any suitable quinone, such as, for example, a benzoquinone, tocoquinone, plastoquinone, coenzyme Q10 or ubiquinone, menaquinone, or phylloquinone, and is further understood to encompass the oxidized or reduced forms.

In a preferred embodiment the composition includes a quinone, and at least one of a solubilizing agent, an emulsifier, and optionally a reducing agent, flavorings, excipients or preservatives contained in a hermetically sealed packet at concentrations that increase the bioavailability, and inhibit recrystallization of CoQ10. In another embodiment of the current invention other biologically active agents or nutrients may be included in the CoQ10 composition to further enhance, for example, the beneficial health effects of CoQ10 or to provide other health benefits when the composition is administered. Nutrients which may be included in the CoQ10 composition include, for example, a fatty acid, a lipid, a carbohydrate, water, an herbal extract, a mineral, a vitamin or any combination thereof.

Pharmaceutically acceptable excipients or carriers may be used to create a suitable form or consistency for administration to an individual, and may include, for example, dicalcium phosphate, silicon dioxide, modified cellulose gum, stearic acid, magnesium stearate, modified cellulose or any combination thereof. In addition, non-ionic detergents and surfactants, for example, polysorbate 80, quillaja extract or the like could be added to increase bioavailability.

In one aspect, the nutritional composition comprises an oil-in-water emulsion. To reduce the amount of oxygen present in the composition, the water may be made substantially air free, for example through boiling under conditions of negative pressure, such as under a vacuum. The oil-in-water emulsion is made using any appropriate technique for producing an oil-in-water emulsion known to those skilled in the art. Preferably, the emulsion is made in an inert atmosphere to prevent oxygen from being entrained in the emulsion. For example, the emulsion may be produced in a nitrogen atmosphere.

In another aspect, the nutritional composition comprises an amount of CoQ10 suitable for a single daily dose and an essential oil disposed in a hermetically sealed container that is substantially free of oxygen. In a particular embodiment, the composition comprises from about 90% to about 99% reduced CoQ10, and an essential oil disposed in a hermetically sealed container which is substantially impermeable to oxygen.

The nutritional composition preferably comprises from about 2% to about 90% by weight CoQ10, from about 10% to about 80% of an oil, for example, an essential oil; and optionally, from about 1% to about 20% of an additional component for example a carrier, an excipients, antioxidant, flavor enhancers, additional active ingredients or nutrients or combinations thereof, the remaining percentage to be made up by water or other solvent.

In another embodiment, the nutritional composition contains from about 20% to about 70% of an oil, for example, an essential oil; from about 1.5% to about 10% by weight CoQ10; and optionally, from about 5% to about 15% of an emulsifier or solubilizer, the remaining percentage to be made up by water or other solvent. Using the proportions described results in a composition which is more viscous than water alone, and reduces the total volume of the composition required to provide a single daily oral dose of CoQ10. As discussed above, the nutritional composition may contain additional components to impart additional desired characteristics or nutrients to the composition.

In an embodiment of the invention, the nutritional composition contains the following components:

| | | |
|---|---|---|
| 1. Medium Chain Triglycerides | 63% | by weight |
| 2. Water | 24% | by weight |
| 3. Emulsifiers | 9% | by weight |
| 4. Ubiquinone | 2.5% | by weight |

The remainder, up to 100% is made up of flavoring, reducing agents, excipients, and preservatives. The composition of the particularly preferred embodiment weighs about 4 grams and will deliver about 100 mg of CoQ10 in a single dose.

In an embodiment of the invention, the nutritional composition contains the following components:

| | |
|---|---|
| 1. Essential Oil | 30% by weight |
| 4. Ubiquinone | 60% by weight |

The remainder, up to 100% is made up of flavoring, reducing agents, excipients, and preservatives.

It is desirable to prepare the nutritional composition in a manner which minimizes exposure of the CoQ10 to oxygen during preparation and storage. In a preferred embodiment the composition may be packaged in a hermetically sealed container to prevent exposure to oxygen during transportation and storage. Minimizing the oxygen content and exposure of the nutritional composition reduces or prevents oxidation and degradation of the CoQ10 in the composition.

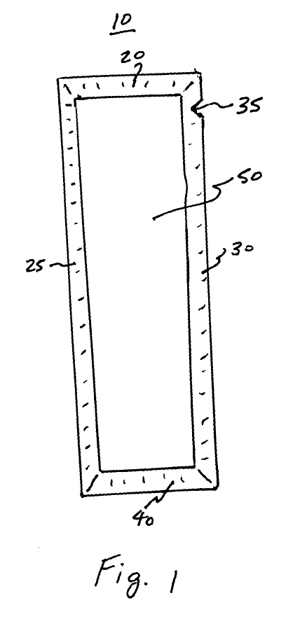

In one embodiment the body portion of the container may be comprised of a flexible, liquid impervious material, for example plastic. In a preferred embodiment the container may be a foil packet similar to those used for transport and storage of food condiments as shown in FIG. 1. The foil packet 10 may be any desired shape. In the embodiment shown in FIG. 1, the packet is generally rectangular in shape. The edges of the packet 20, 25, 30, 40 are heat sealed to hermetically seal the packet. The compositions described above are contained within the packet in a volume 50. The packet may be sized to hold any pre-selected volume of the composition. A notch 35 may be provided at one end of the packet to provide a location for easy tearing of the packet to allow access to the contents of the volume 50.

The invention is not limited in this regard, and any appropriate material or means for providing a hermetically sealed container for the composition may be used. For example, in another embodiment the container is in the form of a sealed bag or a pouch. However, as will be understood by one of ordinary skill, the container may be of any suitable shape, size, or color. Additionally, while the container of the preferred embodiment may be constructed out of flexible plastic or foil, it will be recognized by one of ordinary skill that this is not absolutely required. The container may be constructed of any of various materials, and be any of various degrees of rigidity, so long as the substantial air-resistant transport of a nutritional composition is possible. The container may include any type of suitable opening means located in any portion of the container that is adapted for accessing the nutritional composition.

In further embodiments, the composition comprises a nutritional supplement useful for improving health and treating disease. Research indicates that CoQ10 deficiencies are associated with many diseases and conditions. For example, the composition of the invention is useful for treating, and improving: oxidative phosphorylation, cellular survival, humoral immunity, reduce the oxidation of LDL cholesterol, resistance to viruses, athletic performance, the health of those with diabetes mellitus, and prevent damage to muscles that can occur as a result of intensive exercise.

For example, the composition of the invention is useful for treating, preventing or ameliorating: certain cancers, such as, cervical cancer and its precursor—cervical intraepithelial neoplasia, malignant and nonmalignant breast tumors, arrhythmia, atherosclerosis, hypertension, cardiovascular disease, congestive heart failure, angina, hypoxia, mitral valve prolapse, and progression to full-blown AIDS, allergies, lung cancer, laryngeal cancer, pancreatic cancer, and prostate cancer, proliferation of cancer cells, gingivitis, psoriasis, neuron damage that leads to Alzheimer's disease, and Parkinson's, endothelial function in patients with ischemic heart disease and chronic heart failure.

As will be understood by those of ordinary skill in the pertinent art based on the teachings herein, numerous changes may be made to the above-described and other embodiments of the invention without departing from its scope as defined in the appended claims. For example, the relative quantities of the ingredients may be varied to achieve different desired effects, additional ingredients may be added, and/or similar ingredients may be substituted for one or more of the ingredients described. Accordingly, this detailed description of preferred embodiments is to be taken in an illustrative rather than a limiting sense.

What is claimed is:

1. A composition comprising CoQ10 in a suitable amount to constitute a single daily dose, and a solubilizing agent, wherein said composition is disposed in a hermetically sealed container suitable for transport and storage.

2. The composition of claim 1, wherein the solubilizing agent comprises an essential oil isolated from a plant source selected from the group consisting of lemon, orange, peppermint, spearmint, and anise.

3. The composition of claim 2, wherein the essential oil comprises from about 95% to about 99% D-limonene.

4. A composition comprising from about 2% to about 80% by weight of CoQ10, from about 10% to about 80% by weight of a solubilizing agent, and optionally, from about 1% to about 20% by weight of an emulsifier, the remainder up to 100% selected from the group consisting of a carrier, excipient, antioxidant, reducing agent, preservative, nutrient, water, and combinations thereof, and wherein said composition is disposed in a hermetically sealed container suitable for transport and storage.

5. The composition of claim 4, wherein said solubilizing agent is an oil, an essential oil, a triglyceride, fibersol-2, maltodextrin, polyoxyethanyl-sitosterol sebacate, polyoxyethanyl-cholesteryl sebacate or polyoxyethanyl-.alpha.-tocopheryl sebacate or combination thereof.

6. The composition of claim 5, wherein the solubilizing agent comprises an essential oil isolated from a plant source selected from the group consisting of lemon, orange, peppermint, spearmint, and anise.

7. The composition of claim 6, wherein the essential oil comprises from about 95% to about 99% D-limonene.

8. The composition of claim 4, wherein said emulsifier is a phospholipid, a lecithin, a monoglyceride, casein, a gum, a gum, egg yolk, glyceryl monostearate, ticamulsion or any combination thereof.

9. The composition of claim 4, wherein emulsifier comprises a member from the group consisting of a non-ionic detergent, a surfactant, a fatty acid, a lipid, a carbohydrate, flavoring, a preservative, an herbal extract, a mineral, a vitamin, an excipient and any combination thereof.

10. A composition comprising from about 50% to about 70% by weight of a solubilizing agent, from about 18% to about 30% by weight of water, from about 1.5% to about 10% by weight of ubiquinone, from about 5% to about 15% by weight of an emulsifier, wherein the composition is disposed in a hermetically sealed, air-tight container adapted for receiving, storing, and transporting a suitable volume of said composition for the delivery of a single or divided daily dose.

11. The composition of claim 10, wherein said solubilizing agent is an oil, an essential oil, a triglyceride, fibersol-2, maltodextrin, polyoxyethanyl-sitosterol sebacate, polyoxyethanyl-cholesteryl sebacate or polyoxyethanyl-.alpha.-tocopheryl sebacate or combination thereof.

12. The composition of claim 10, wherein said emulsifier is a phospholipid, a lecithin, a monoglyceride, casein, a gum, a gum, egg yolk, glyceryl monostearate, ticamulsion or any combination thereof.

13. The composition of claim 10, further comprising a suitable amount of a reducing agent.

14. The composition of claim 10, further comprising a member from the group consisting of a non-ionic detergent, a surfactant, a fatty acid, a lipid, a carbohydrate, flavoring, a preservative, an herbal extract, a mineral, a vitamin, an excipient and any combination thereof.

15. An emulsion comprising about 63% by weight of a triglyceride, about 24% by weight of water; about 2.5% by weight of ubiquinone, and about 9% by weight of an emulsifier.

16. The emulsion of claim 15, wherein said emulsifier is a phospholipid, a lecithin, a monoglyceride, casein, a gum, a gum, egg yolk, glyceryl monostearate, ticamulsion or any combination thereof.

17. The emulsion of claim 15, wherein the emulsion further comprises a suitable amount of a reducing agent.

18. The emulsion of claim 15, wherein the emulsion further comprises a hermetically sealed, air-tight container adapted for receiving, storing, and transporting a suitable volume of said composition for the delivery of a single or divided daily dose.

19. The emulsion of claim 15, wherein the emulsion further comprises a member from the group consisting of a non-ionic detergent, a surfactant, a fatty acid, a lipid, a carbohydrate, flavoring, a preservative, an herbal extract, an essential oil, a mineral, a vitamin, an excipient and any combination thereof.
